(19) United States
(12) Patent Application Publication
(10) Pub. No.: US 2012/0277464 A1
(43) Pub. Date: Nov. 1, 2012

(54) METHOD AND DEVICE FOR PRODUCING ALKENE DERIVATIVES

(75) Inventors: Christophe Claeys, Veron (FR); Alberto Garcia, Valladolid (ES); Sylvain Gerard, Saint-Cloud (FR); Nicolas Dupont, Metz (FR); Jean Luc Dubois, Millery (FR); Serge Tretjak, Roulhing (FR); Nabil Tlili, Mulhouse (FR)

(73) Assignees: Arkema France, Colombes (FR); L'Air Liquide Societe Anonyme Puor L'Exploitation des Procedes Georges Claude, Paris (FR)

(21) Appl. No.: 13/505,325

(22) PCT Filed: Oct. 27, 2010
(86) PCT No.: PCT/FR10/52302

§ 371 Date: Jul. 17, 2012

(30) Foreign Application Priority Data
Nov. 2, 2009 (FR) 0957731

(51) Int. Cl. C07C 51/25; B01J 19/00; C07C 45/35

(52) U.S. Cl. 562/512.2; 568/469.9; 422/187

(57) ABSTRACT

The invention relates to a method for producing a flow containing at least one alkene derivative, including the following steps: a step a) of reacting a flow containing one or more alkenes and one or more alkanes—the ratio of said alkanes to said alkenes being at least 1 by volume—with a flow containing mainly oxygen, in order to obtain at least one converted flow containing at least said alkene derivative; a step b) of separating the converted flow produced in step a) into at least said flow containing at least said alkene derivative and a residual flow containing mainly one or more hydrocarbons and one or more inert compounds; and a step c) of separating all or a portion of said residual flow by means of permeation into at least one first flow containing mainly one or more inert compounds and a second flow containing mainly one or more hydrocarbons.

The invention relates to a process and a device for the production of alkene derivatives.

Processes for the production of alkene derivatives are generally fed with alkene feedstocks of very good purity often greater than 95% by weight, in order to minimize the operations for separation of products downstream of the conversion process. This purity is generally obtained by purification of mixtures of alkanes and alkenes of lower purity, generally by distillation or liquid-liquid extraction. The corresponding purification units represent significant capital and operation costs, in particular due to the small difference in the physical properties (for example the volatility, in the case of a separation by distillation) of hydrocarbons to be separated.

The document U.S. Pat. No. 6,667,409 describes the incorporation of the process of production of alkenes from alkanes in the process of the production of the alkene derivatives. It discloses an alkanes/alkenes separation in order to obtain a feedstock enriched in alkenes which is sent to the unit for the production of alkene derivatives. This prior separation is expensive in terms of energy.

The documents U.S. Pat. No. 4,532,365 and FR-A-2 525 212 describe the dehydrogenation of an alkane to form a mixture comprising the corresponding alkene, hydrogen, carbon oxides and the unreacted alkane. This mixture, to which oxygen is added, is sent over an oxidation catalyst in order to produce an alkene derivative, for example acrolein. After recovering this derivative by absorption, the gas stream exiting from the absorber is recycled to the dehydrogenation stage. In point of fact, this requires removing the oxygen from the stream, which is obtained by reacting the oxygen and the hydrogen over a catalyst. The carbon oxides are absorbed by a washing solution (e.g. amines or carbonates). The separation of carbon oxides by physicochemical washing consumes a great deal of energy during the regeneration phase and produces waste (decomposed amines, carbonates) to be treated and discarded. In addition, the separation process employed in the document U.S. Pat. No. 4,532,635 does not make it possible to recover the non-condensable compounds, such as oxygen. The latter can no longer be recycled and is consequently lost, which has an impact on the global cost of the process.

The document US 2006/004226 A1 discloses a process for the production of acrolein or acrylic acid from propane. The propane is dehydrogenated by heterogeneous catalysis, the secondary components are separated and the gas mixture, comprising propane, and propene is partially oxygenated by heterogeneous catalysis to form a stream comprising the product. The latter is separated into a product stream and another stream comprising the unconverted propane and the excess oxygen. This stream is recycled to the dehydrogenation stage without additional separation.

The document U.S. Pat. No. 6,423,875 also discloses a process for the production of an acrolein or acrylic acid derivative from a feedstock comprising propane, and air, by virtue of an oxydehydrogenation of the propane with the air to form a mixture comprising propylene. This mixture is subsequently directed to a gas-phase oxidation process to form a stream comprising the product. The latter is separated into a product stream and another stream comprising unconverted propane and inert compounds. This stream is recycled to the oxydehydrogenation stage. It is freed beforehand by cryogenic distillation from nitrogen and all the constituents having a boiling point below the boiling point of the propylene. The distillation employed is expensive and raises questions of safety as the mixture to be distilled comprises hydrocarbons and oxygen. Furthermore, the use of air as oxidant limits the productivity of the whole process.

The document U.S. Pat. No. 5,646,304 describes an oxidation of alkenes by pure oxygen, with recirculation of the unconverted compounds, i.e. hydrocarbons, after separation by an adsorption process of PSA type, TSA type or a combination of the two. These processes are semi-batchwise, which is a source of complexity in their control. They require numerous valves and wear equipment calling for expensive maintenance. The regeneration of the adsorbers requires an external gas and creates effluent streams which have to be treated. As the purge of the PSA/TSA process has a low NCV (net calorific value), it is incinerated by adding a fuel having a high NCV, such as natural gas. There also exists risks of over-concentration of oxygen in the presence of hydrocarbons in the adsorbers, during the recompression phases in particular. If the adsorbent products are active charcoals, the risks become totally unacceptable in terms of safety and flammability. In addition, argon tends to accumulate in the overall process.

One aim of the present invention is to overcome all or part of the abovementioned disadvantages, that is to say in particular to provide a process and device for the production of alkene derivatives which can be fed with a feedstock of alkenes of low purity in an efficient way in terms of energy consumption and productivity, without major capital costs and under good safety conditions.

To this end, the invention relates to a process for the production of a stream comprising at least one alkene derivative comprising the following stages:
a stage a) of reaction of a stream comprising one or more alkenes and one or more alkanes, the ratio of said alkanes to said alkenes being at least equal to 1 by volume, with a stream predominantly comprising oxygen, in order to obtain at least one converted stream comprising at least said alkene derivative;
a stage b) of separation of the converted stream resulting from stage a) into at least said stream comprising at least said alkene derivative and a residual stream predominantly comprising one or more hydrocarbons and one or more inert compounds; and
a stage c) of separation by permeation of all or part of said residual stream into at least one first stream predominantly comprising one or more inert compounds and a second stream predominantly comprising one or more hydrocarbons.

“Predominantly” should be understood as meaning, here as in the whole of the present document, at least 50% by volume. Stream is understood to mean a certain amount of fluid, per unit of time, it being possible for the fluid to be liquid, gaseous or two-phase. The present invention relates in particular to gas-phase streams. Hydrocarbons is understood to mean alkane, alkene or a mixture comprising at least one alkane and at least one alkene.

In stage a), one of the streams which reacts comprises one or more alkenes and at least as much alkane by volume, that is to say that the ratio by volume of the alkanes to the alkenes is at least equal to 1. The alkenes in question can in particular be ethylene, propylene or isobutene. The alkanes are unreactive or less reactive compounds in the chemical reactions involved during the conversion, and can be methane, ethane, propane, or isobutane, for example. The other components of this alkene stream can be compounds which are inert in these same reactions, such as nitrogen or argon. The other compounds can also comprise water, CO or CO₂. The reaction takes place not with air, but with a stream predominantly comprising oxygen. Preferably, this stream is gaseous and comprises at least 90% of oxygen by volume. Consequently, much less nitrogen is introduced into the conversion unit than if air were used.

The non-reactional part of the stream is denoted by the term “gas ballast”. The constituents of the gas ballast do not participate in the chemical reactions. Their interest lies, on the other hand, in their heat capacity (Cp), i.e. their ability to capture the heat released by the chemical reaction while limiting the increase in the temperature.

Preferably, the gas ballast comprises less than 10%, indeed, even less than 5%, by volume of a gas chosen from nitrogen, argon and their mixtures. The large gas ballast formed by said alkanes exhibits several advantages in comparison with a nitrogen ballast. First, it creates a better thermal ballast as its specific heat capacity (Cp) strongly increases with the temperature, which is not the case with nitrogen. In addition, it has a certain chemical inertia under the conditions of the reaction carried out in stage a); furthermore, if it reacts at stage a), the reaction products are very similar in nature to those which would be obtained from a feedstock of alkenes devoid of alkanes. Finally, it makes it possible to more easily meet the constraints of composition of the mixture related to the question of the inflammability by moving the reaction mixture above the upper explosive limit. By virtue of the novel properties of this ballast in comparison with those of a ballast predominantly comprising nitrogen, the feedstock feeding stage a) can comprise more alkenes as fraction by volume, which increases the productivity of the conversion. Specifically, a greater part of the heat of reaction can be captured by the gas ballast for the same temperature in the reactor. Finally the thermal properties of this ballast make it possible to exert better control of the hot spots in the bed of catalysts and thus to promote the selectivity of the reaction.

The conversion produces at least one converted stream comprising at least said alkene derivative which it is desired to produce. The alkene derivatives in particular can be ethylene oxide, acrolein, acrylic acid, methacrolein or methacrylic acid. The applications may cover generally all gas phase oxidations of alkenes comprising from 2 to 4 carbons. The other components of the converted stream generally comprise other compounds, such as CO, CO₂, water, nitrogen and/or argon, and hydrocarbons which are not reacted, or not completely reacted in the conversion unit. The mixture of the alkanes and the other compounds of the converted stream constitutes a thermal gas ballast exhibiting the abovementioned advantages.

Stage a) can be employed in a multitube fixed bed reactor or a fluidized bed reactor or a circulating fluidized bed reactor or plate reactors.

In stage b), the conversion stream is separated into at least one stream comprising the alkene derivative or derivatives which it is desired to produce and a residual stream comprising said gas ballast and the inert compounds. This separation can be carried out by absorption of the alkene derivatives in one or more solvents, for example water. For this stage, use may be made, for example of an absorption column in which the stream resulting from stage a) encounters, countercurrentwise, a solvent introduced at the column top or obtained by partial condensation of the light compounds (for example water) present in the gas phase.

In stage c), this residual stream, in all or in part, is separated in a selective permeation unit into at least one first stream predominantly comprising the abovementioned inert compounds and a second stream predominantly comprising hydrocarbons. The latter is generally recycled in order to be employed in stage a) and/or is used in another unit (dehydrogenation of alkanes, hydrocarbons cracker, and the like) and/or is simply used as fuel (boiler furnace). The permeation unit employs one or more semipermeable membranes having the property of retaining certain compounds and, on the contrary, of allowing others of them to pass. Depending on the purities desired, it may prove to be necessary to use several purification stages. The separation by permeation generally takes place at a pressure of the order of 10 bar (1 bar=0.1 MPa) and at a temperature of approximately 50° C. This type of membrane separation can be carried out by virtue of products based on hollow fibers composed of a polymer chosen from: polyimides, polymers of cellulose derivatives type, polysulfones, polyamides, polyesters, polyethers, polyetherketones, polyetherimides, polyethylenes, polyacetylenes, polyethersulfones, polysiloxanes, polyvinylidene fluorides, polybenzimidazoles, polybenzoxazoles, polyacrylonitriles, polyazoaromatics and the copolymers of these polymers.

One advantage of the process according to the invention is that it can be fed with a feedstock where the ratio by volume of the alkanes to the alkenes is at least equal to 1. The compounds which are not alkenes form a gas ballast mainly composed of alkanes. In general, the gas ballast comprises at least 30% by volume of alkanes, preferably at least 50% by volume of alkanes. This feed generally originates from a column for the fractionation of alkanes/alkenes, from a steam cracker or catalytic cracker (optionally followed by hydrogenation of the diolefins), from a unit for the dehydrogenation of alkanes or from the recycling of the gas ballast. The alkenes/alkanes feedstock is sent directly to the unit for conversion into alkene derivatives. The separation of the alkanes and inert compounds (e.g. CO₂) by permeation after the unit for the production of alkene derivatives exhibits the advantage of being more efficient energetically than the conventional separation of the alkenes and alkanes upstream of the conversion unit targeted at obtaining a feedstock of alkenes of high purity and a stream of alkanes. In other words, the process according to the invention, using a feedstock of alkenes of low purity and a permeation unit, makes it possible to produce alkene derivatives and a stream rich in alkanes at a lower energy cost than a conventional process using a feedstock of alkenes of high purity and not employing a permeation stage.

Unlike the process as described in the document U.S. Pat. No. 4,532,635, the process according to the invention, which comprises a stage of separation by permeation, makes possible the recovery of most of the oxygen and the removal of the carbon oxides and argon, while avoiding recourse to an additional energy-consuming unit operation.

The separation by permeation of the present invention makes it possible to selectively separate the inert compounds from the hydrocarbons at a lower operating cost than a cryogenic distillation, such as that described in document U.S. Pat. No. 6,423,875. This is because the pressure and the temperature which are necessary for the separation by permeation are typically 10 bar (1 bar=0.1 MPa) and 50° C., whereas the cryogenic distillation as described in U.S. Pat. No. 6,423,875 requires a pressure of greater than 50 bar and, by definition, cryogenic temperatures. Moreover, the separation by permeation exhibits the advantage of being continuous, of not requiring a regeneration stage (consumption of external gas and the production of effluents to be treated), of not presenting the risks related to an excess concentration of oxygen of the adsorption processes and, finally, of making it possible to purge the argon, which is regarded as thermal poison as a result of its low specific heat and which, if it accumulated in the process, would damage the thermal properties of the gas ballast.

In addition, this makes it possible to reduce the size and thus the cost of the alkenes/alkanes separation process located upstream (for example a fractionation column), and even to eliminate it if the process for production of alkene derivatives is the only process using this feedstock. The operating costs (energy to separate the alkanes from the alkenes) of the fractionation column are also considerably reduced. Another advantage lies in the fact of carrying out an oxidation reaction in a gas stream rich in alkanes, which constitutes a thermal ballast. This oxidation ideally will be carried out by virtue of a stream predominantly comprising oxygen (at least 50% by volume), preferably at least 90%, in order to minimize the presence of nitrogen (or other inert compounds) and to benefit from the advantages of a thermal ballast mentioned above.

According to specific embodiments, the invention can comprise one or more of the following characteristics:
a non-zero proportion of said second stream predominantly comprising one or more hydrocarbons is employed in stage a). This makes it possible to increase the fraction by volume of ballast in the feedstock of alkenes converted in stage a). This is because this feedstock generally originates from a column for the fractionation of alkanes/alkenes, from a steam cracker or catalytic cracker (optionally followed by hydrogenation of the diolefins) or from a unit for the dehydrogenation of alkanes. The ratio by volume of the alkanes to the alkenes is then generally between 1/20 and 20. The recycling of said second stream predominantly comprising one or more hydrocarbons in this feedstock makes it possible to adjust upwardly the ratio by volume of the alkanes to the alkenes. In stage a), before conversion, the ratio by volume of alkanes to alkenes must be at least equal to 1.
said stream comprising one or more alkenes and one or more alkanes employed in stage a) comprises from 2% to 20% of alkenes by volume. Preferably said stream comprising one or more alkenes and one or more alkanes employed in stage a) comprises at least 20% of alkanes.
the process additionally comprises a stage d) prior to stage a) of reaction of a stream predominantly comprising one or more alkanes with a stream predominantly comprising oxygen in order to obtain at least said stream comprising one or more alkenes and one or more alkanes employed in stage a).
a non-zero proportion of the second stream, obtained in stage c), predominantly comprising one or more hydrocarbons, is reacted in stage d).
the process additionally comprises a stage f), adjacent and prior to stage c), of catalytic oxidation of carbon monoxide, optionally present in said residual stream predominantly comprising one or more hydrocarbons and one or more inert compounds, to give carbon dioxide. Stage f) can relate to all or part of said residual stream.
the process additionally comprises a stage g), adjacent and subsequent to stage c), of catalytic oxidation of carbon monoxide, optionally present in said second stream predominantly comprising one or more hydrocarbons, to give carbon dioxide. Stage g) can relate to all or part of said second stream predominantly comprising one or more hydrocarbons.
said alkenes predominantly comprise, and preferably are, propylene, said alkanes predominantly comprise, and preferably are, propane and said alkene derivative is acrolein and/or acrylic acid.

In a specific embodiment of the invention, a non-zero fraction of the second stream resulting from the membrane separation, predominantly comprising hydrocarbons, typically alkanes, can be employed in stage a). “Non-zero fraction” is understood to mean any fraction greater than 0% and which can range up to 100%. “Employed” means that the stream fraction in question participates in the reaction either as reactant, or as passive compound, with a possible role of thermal or chemical ballast.

The alkenes of low purity to be converted can originate from a process for the oxydehydrogenation, or oxidative dehydrogenation or dehydrogenation of the corresponding alkane. This makes possible a partial conversion of the alkane to the corresponding alkene and makes it possible to provide a process for the conversion of the alkenes with a feedstock rich in alkane. The unconverted hydrocarbons resulting from the separation by permeation can be employed in the units for the oxidative or non-oxidative dehydrogenation of the alkanes. However, the second stream predominantly comprising hydrocarbons, which is rich in alkanes, will instead be sent directly to the reaction for the conversion of the alkenes in stage a), or to other processes (cracking, furnace and the like), in order to dispense with any other subsequent purification after the separation by permeation targeted at preventing any contamination or side reactions in the process for the oxidative or non-oxidative dehydrogenation of the alkanes.

The oxidation of CO which is the object of the stages f) and g) can also be carried out in parallel with the membrane separation, the separation taking place in the next cycle. This makes it possible, in this case to minimize the size of the CO converter.

The invention also relates to a plant for the production of a stream comprising at least one alkene derivative, said plant comprising:
a unit for conversion of alkenes to alkene derivatives;
a source of a stream comprising from 2% to 20% by volume of one or more alkenes connected fluidically to said conversion unit;
a source of a stream predominantly comprising oxygen connected fluidically to said conversion unit;
a separator connected fluidically to an outlet of said conversion unit; and
a unit for separation by permeation connected fluidically to an outlet of said separator.

“Fluidic connection” or “connected fluidically” means that there is connection via a system of pipes capable of transporting a stream of material. This connection system can comprise valves, intermediate storage tanks, side outlets, heat exchangers and compressors but not chemical reactors.

According to specific embodiments, the invention can comprise one or more of the following characteristics:
it comprises a fluidic connection between an outlet of said unit for separation by permeation and said conversion unit or said source of a stream predominantly comprising one or more alkenes.
it comprises a fluidic connection between an outlet of said separator and an outlet of said unit for separation by permeation.
said source of a stream comprising from 2% to 20% by volume of one or more alkenes comprises a:
   oxydehydrogenation reactor connected fluidically to a source of a stream predominantly comprising one or more alkanes and to a source of a stream predominantly comprising oxygen; or
   dehydrogenation reactor connected fluidically to a source of a stream predominantly comprising one or more alkanes.
the plant comprises recycling means placed between an outlet of said unit for separation by permeation and said oxydehydrogenation or dehydrogenation reactor, or said source of a stream predominantly comprising one or more alkanes.
the plant comprises a unit for the catalytic conversion of carbon monoxide to carbon dioxide connected fluidically to said separator and to said unit for separation by permeation.
the plant comprises a unit for the catalytic conversion of carbon monoxide to carbon dioxide connected fluidically to an outlet of said unit for separation by permeation.

The optional recycling upstream of the oxydehydrogenation or dehydrogenation reactor can take place in the source of alkanes or in the stream which emerges therefrom (between the source and the oxidative dehydrogenation reactor) or else directly in the oxidative dehydrogenation reactor.

### BRIEF DESCRIPTION OF THE DRAWINGS

Other distinctive features and advantages will become apparent on reading the description below, made with reference to FIG. 1, which represents a diagrammatic and partial view illustrating an example of the device according to the invention.

FIG. 1 shows a reactor 15 which produces alkenes of low purity (2 to 98%), for example propylene and a propane ballast. It provides a feedstock 23 which, mixed with a recycled stream 10, forms a stream 1 injected into a unit 2 for the production of acrylic acid by oxidation of propylene by oxygen which is 99% pure by volume. The stream 3 comprising acrylic acid exits therefrom. This stream 3 is separated in a separator 4 into a stream 5 of acrylic acid and a stream mainly comprising propane and CO₂. This stream is directed partially (stream 6 to a separating unit 7 and stream 24 which by-passes the separating unit) or completely (stream 6) to unit 7 of separation by permeation. Beforehand, a condensation can make it possible to separate the water (mainly) from the other constituents (stream 27). The separation unit 7 comprises membranes based on hollow polyetherimide fibers, in a number sufficient to purge, in the stream 8, the amount of CO₂ produced in the unit 2 and the units 21 and 22. The stream 6 feeds, at high pressure (for example 10 bar), the unit 7 comprising the semipermeable membranes. The compounds present in the stream 6 will be dissolved and will diffuse through the polymer fibers of the unit 7 at different rates, so that, preferentially, the fast compounds will pass through the fiber and will be reencountered on the low-pressure side of the membrane, known as permeate stream and constituting the purge. The slow compounds will, for their part, remain on the high-pressure side and will constitute the stream 9. The stream 8 comprises CO₂ (the main inert compound which has to be purged from the stream 6) and the stream 9 comprises propane. A non-zero fraction 10 is mixed with a stream 23 to form the stream 1 sent to the conversion unit 2. A non-zero fraction 11 of the stream 9 is sent to one or more user units and/or is simply used as fuel.

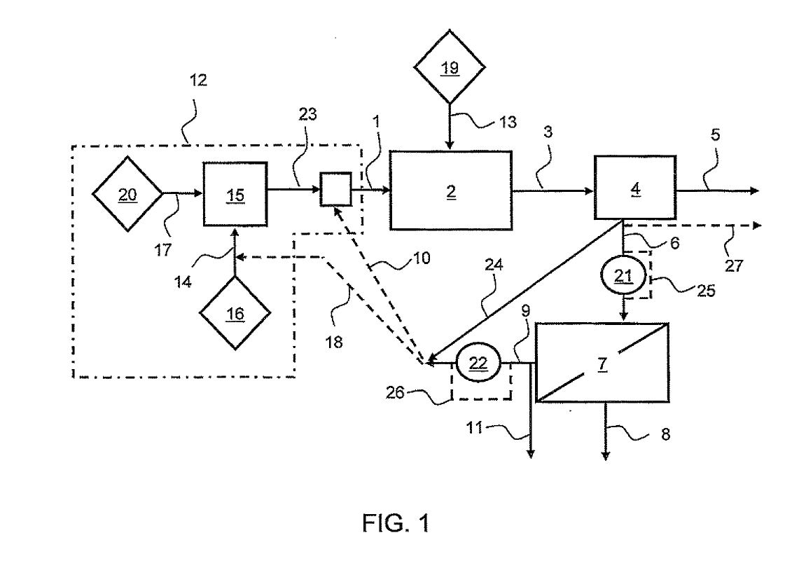

The reactor 15 carries out an oxydehydrogenation of a stream 14 comprising propane originating from a source 16. This oxydehydrogenation requires a stream 17 predominantly comprising oxygen from a source 20. A non-zero fraction 18 of the stream 9 is injected into the stream 14 or else directly into the oxydehydrogenation reactor 15. The stream 23 comprises propylene and propane. The combination 15, 16, 20 forms part of a source 12 supplying the stream 1.

The stream 6, prior to its entry into the unit 7 for separation by permeation is treated in a unit 21 for catalytic conversion of carbon monoxide to carbon dioxide. An alternative to this process considers the catalytic conversion of carbon monoxide to carbon dioxide in the stream 9 by the unit 22 and not in the stream 6 by the unit 21. It is not inevitably necessary to carry out this catalytic conversion (units 21 and 22) with regard to the whole of the streams 6 and 9. A by-pass is thus possible, represented diagrammatically by the streams 25 and 26. It is also possible to have a by-pass in the form of a stream parallel to the stream 24 and equipped with a CO converter.

### EXAMPLE 1

### Carbon Monoxide Converter (21) Placed Upstream of the Separation Unit (7)

The molar compositions of the main streams generated under the molar flow rate, pressure and temperature conditions mentioned are shown in table 1.

TABLE 1

| | | 23 | 1 | 13 | 5 | 6 | 8 | 9 |
|---|---|---|---|---|---|---|---|---|
| Acetaldehyde | mol % | 0.00% | 0.09% | 0.00% | 0.00% | 0.11% | 0.00% | 0.14% |
| Acrolein | mol % | 0.00% | 0.14% | 0.00% | 0.00% | 0.24% | 0.00% | 0.06% |
| H₂O | mol % | 0.00% | 14.18% | 0.00% | 62.71% | 2.43% | 3.01% | 8.57% |
| O₂ | mol % | 0.00% | 5.27% | 99.50% | 0.00% | 10.26% | 1.95% | 0.79% |
| Argon | mol % | 0.00% | 1.38% | 0.50% | 0.00% | 1.99% | 2.58% | 1.85% |
| CO | mol % | 0.00% | 2.05% | 0.00% | 0.00% | 3.95% | 0.38% | 0.40% |
| CO₂ | mol % | 0.00% | 16.02% | 0.00% | 0.00% | 23.90% | 82.65% | 19.64% |
| Propane | mol % | 5.50% | 41.62% | 0.00% | 0.00% | 53.87% | 8.29% | 66.62% |
| Propylene | mol % | 94.50% | 17.95% | 0.00% | 0.00% | 1.67% | 0.08% | 0.19% |
| Acetic acid | mol % | 0.00% | 0.02% | 0.00% | 1.63% | 0.00% | 0.00% | 0.00% |
| Acrylic acid | mol % | 0.00% | 0.07% | 0.00% | 34.54% | 0.01% | 0.00% | 0.02% |
| Others | mol % | 0.00% | 1.21% | 0.00% | 1.11% | 1.56% | 1.05% | 1.72% |
| Flowrate | mol/h | 32.67 | 182.7 | 56.24 | 78.12 | 49.14 | 10.22 | 38.47 |
| Pressure | bar | 7.5 | 2.25 | 3.6 | 1 | 3 | 2 | 11.5 |
| Temperature | ° C. | 60 | 153 | 20 | 80 | 40 | 50 | 50 |

### EXAMPLE 2

### Carbon Monoxide Converter (21) Placed Downstream of the Separation Unit (7)

The molar compositions of the main streams generated under the molar flow rate, pressure and temperature conditions mentioned are shown in table 2.

TABLE 2

| | | 23 | 1 | 13 | 5 | 6 | 8 | 9 |
|---|---|---|---|---|---|---|---|---|
| Acetaldehyde | mol % | 0.00% | 0.09% | 0.00% | 0.00% | 0.10% | 0.00% | 0.12% |
| Acrolein | mol % | 0.00% | 0.24% | 0.00% | 0.00% | 0.25% | 0.00% | 0.31% |
| H₂O | mol % | 0.00% | 16.03% | 0.00% | 63.03% | 2.43% | 1.31% | 2.68% |
| O₂ | mol % | 0.00% | 3.99% | 99.50% | 0.00% | 5.80% | 12.22% | 4.39% |
| Argon | mol % | 0.00% | 1.79% | 0.50% | 0.00% | 2.12% | 2.92% | 1.95% |
| CO | mol % | 0.00% | 3.79% | 0.00% | 0.00% | 5.31% | 5.33% | 5.31% |
| CO₂ | mol % | 0.00% | 18.46% | 0.00% | 0.00% | 23.09% | 67.69% | 13.34% |
| Propane | mol % | 5.50% | 52.91% | 0.00% | 0.00% | 57.97% | 8.73% | 68.73% |
| Propylene | mol % | 94.50% | 1.50% | 0.00% | 0.00% | 1.74% | 0.88% | 1.93% |
| Acetic acid | mol % | 0.00% | 0.02% | 0.00% | 1.57% | 0.00% | 0.00% | 0.00% |
| Acrylic acid | mol % | 0.00% | 0.08% | 0.00% | 34.30% | 0.01% | 0.00% | 0.01% |
| Others | mol % | 0.00% | 1.10% | 0.00% | 1.10% | 1.18% | 0.92% | 1.24% |
| Flow rate | mol/h | 32.08 | 174.81 | 53.574 | 78.66 | 47.32 | 8.49 | 36.57 |
| Pressure | bar | 7.5 | 2.25 | 3.6 | 1 | 3 | 2 | 11.5 |
| Temperature | ° C. | 60 | 153 | 20 | 80 | 40 | 50 | 50 |

In the tables, the pressures are in bars absolute.

1. A process for the production of a stream (5) comprising at least one alkene derivative comprising the following stages:
a stage a) of reaction (2) of a stream (1) comprising one or more alkenes and one or more alkanes, the ratio of said alkanes to said alkenes being at least equal to 1 by volume, with a stream (13) predominantly comprising oxygen, in order to obtain at least one converted stream (3) comprising at least said alkene derivative;
a stage b) of separation (4) of the converted stream (3) resulting from stage a) into at least said stream (5) comprising at least said alkene derivative and a residual stream (6) predominantly comprising one or more hydrocarbons and one or more inert compounds; and
a stage c) of separation by permeation (7) of all or part of said residual stream (6) into at least one first stream (8) predominantly comprising one or more inert compounds and a second stream (9) predominantly comprising one or more hydrocarbons.

2. The process as claimed in claim 1, characterized in that a non-zero proportion (10) of said second stream (9) predominantly comprising one or more hydrocarbons is employed in stage a).

3. The process as claimed in claim 1, characterized in that said stream (1) comprising one or more alkenes and one or more alkanes employed in stage a) comprises from 2% to 20% of alkenes by volume.

4. The process as claimed in claim 1, characterized in that it additionally comprises a stage d) prior to stage a) of reaction of a stream (14) predominantly comprising one or more alkanes with a stream (17) predominantly comprising oxygen in order to obtain at least said stream (1) comprising one or more alkenes and one or more alkanes employed in stage a).

5. The process as claimed in claim 4, characterized in that a non-zero proportion (18) of the second stream (9), obtained in stage c), predominantly comprising one or more hydrocarbons, is reacted in stage d).

6. The process as claimed in claim 1, characterized in that it additionally comprises:
a stage f), adjacent and prior to stage c), of catalytic oxidation (21) of carbon monoxide, optionally present in said residual stream (6) predominantly comprising one or more hydrocarbons and one or more inert compounds, to give carbon dioxide; and/or
a stage g), adjacent and subsequent to stage c), of catalytic oxidation (22) of carbon monoxide, optionally present in said second stream (9) predominantly comprising one or more hydrocarbons, to give carbon dioxide.

7. The process as claimed in claim 1, characterized in that said alkenes predominantly comprise, and preferably are, propylene, said alkanes predominantly comprise, and preferably are, propane and said alkene derivative is acrolein and/or acrylic acid.

8. A plant for the production of a stream (5) comprising at least one alkene derivative, said plant comprising:
a unit for conversion (2) of alkenes to alkene derivatives;
a source (12) of a stream comprising from 2% to 20% by volume of one or more alkenes connected fluidically (1) to said conversion unit (2);
a source (19) of a stream predominantly comprising oxygen connected fluidically (13) to said conversion unit (2);
a separator (4) connected fluidically to an outlet (3) of said conversion unit (2); and
a unit for separation by permeation (7) connected fluidically to an outlet (6) of said separator (4).

9. The plant as claimed in claim 8, characterized in that it comprises a fluidic connection (10) between an outlet (9) of said unit for separation by permeation (7) and said conversion unit (2) or said source (12) of a stream predominantly comprising one or more alkenes.

10. The plant as claimed in claim 8, characterized in that it comprises a fluidic connection (24) between an outlet of said separator (4) and an outlet (9) of said unit for separation by permeation (7).

11. The plant as claimed in claim 8, characterized in that said source (12) of a stream comprising from 2% to 20% by volume of one or more alkenes comprises a:
oxydehydrogenation reactor (15) connected fluidically (14) to a source (16) of a stream predominantly comprising one or more alkanes and to (17) a source (20) of a stream predominantly comprising oxygen; or
dehydrogenation reactor (15) connected fluidically (14) to a source (16) of a stream predominantly comprising one or more alkanes.

12. The plant as claimed in claim 11, characterized in that it comprises recycling means (18) placed between an outlet (9) of said unit for separation by permeation (7) and said oxydehydrogenation or dehydrogenation reactor (15), or said source (16) of a stream predominantly comprising one or more alkanes.

13. The plant as claimed in claim 8, characterized in that it comprises:
a unit (21) for the catalytic conversion of carbon monoxide to carbon dioxide connected fluidically to said separator (4) and to said unit for separation by permeation (7); and/or
a unit (22) for the catalytic conversion of carbon monoxide to carbon dioxide connected fluidically to an outlet (9) of said unit for separation by permeation (7).
